(19) United States
(12) Patent Application Publication
(10) Pub. No.: US 2006/0051872 A1
(43) Pub. Date: Mar. 9, 2006

(54) Synthesis and use of inorganic polymer sensor for detecting nitroaromatic compounds

(75) Inventors: Michael J Sailor, La Jolla, CA (US); William C Trogler, Del Mar, CA (US); Honglae Sohm, Gwangju (KR); Sarah J Toal, La Jolla, CA (US)

Correspondence Address: GREER, BURNS & CRAIN, 300 S WACKER DR, 25TH FLOOR, CHICAGO, IL, 60606, US

(73) Assignee: The Regents of Califorina, Oakland, CA (US)

(21) Appl. No.: 10/528,967

(22) PCT Filed: Oct. 6, 2003
(86) PCT No.: PCT/US03/31600

§ 371 Date: Jul. 26, 2005

Related U.S. Application Data
(60) Provisional application No. 60/416,292, filed on Oct. 5, 2002.
(60) Provisional application No. 60/416,289, filed on Oct. 5, 2002.

(51) Int. Cl. C07F 7/08; C07F 7/04; G01N 33/00

(52) U.S. Cl. 436/86; 556/1; 556/431; 528/10

(57) ABSTRACT

A dehydrocoupling polycondensation method for synthesizing polymetalloles including obtaining a dihydrometallole that includes silicon or germanium atoms, designating a reducing agent for preparation of dihydrometallole monomer, measuring a predetermined molar percentage of the reducing agent corresponding to a molar amount of the dihydrometallole, selecting a catalyst, and reacting the catalyst with the dihydrometallole to obtain a polymetallole. A method for detecting an analyte that may be present in ambient air or complex aqueous media including providing a polymer or copolymer containing a metalloid-metalloid backbone, exposing the polymer or copolymer to a suspected analyte or a system suspected of including the analyte, and measuring a quenching of photoluminescence of the metallole polymer or copolymer exposed to the system.

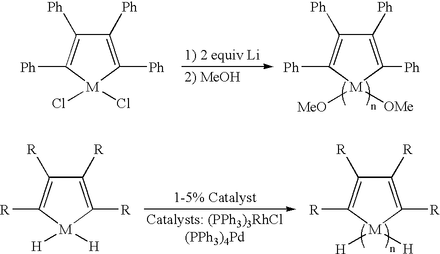

### TECHNICAL FIELD

A field of the invention is analyte detection. The instant invention is directed to the synthesis and use of inorganic polymers, namely photoluminescent metallole polymers and copolymers, for detection of nitroaromatic compounds based on photoluminescence quenching.

### BACKGROUND ART

Use of chemical sensors to detect ultra-trace analytes from explosives has been the focus of investigation in recent years owing to the critical importance of detecting explosives in a wide variety of areas, such as mine fields, military bases, remediation sites, and urban transportation areas. Detecting explosive analytes also has obvious applications for homeland security and forensic applications, such as the examination of post-blast residue. Typically these chemical sensors are small synthetic molecules that produce a measurable signal upon interaction with a specific analyte.

Chemical sensors are preferable to other detection devices such as metal detectors because metal detectors frequently fail to detect explosives, such as in the case of the plastic casing of modern land mines. Similarly, trained dogs are both expensive and difficult to maintain. Other detection methods, such as gas chromatography coupled with a mass spectrometer, surface-enhanced Raman, nuclear quadrupole resonance, energy-dispersive X-ray diffraction, neutron activation analysis and electron capture detection are highly selective, but are expensive and not easily adapted to a small, low-power package.

Conventional chemical sensors have drawbacks as well. Sensing TNT and picric acid in groundwater or seawater is important for the detection of buried, unexploded ordnance and for locating underwater mines, but most chemical sensor detection methods are only applicable to air samples because interference problems are encountered in complex aqueous media. Thus, conventional chemical sensors are inefficient in environmental applications for characterizing soil and groundwater contaminated with toxic TNT at military bases and munitions production and distribution facilities. Also, conventional chemical sensors, such as highly π-conjugated, porous organic polymers, are commonly used as chemical sensors and can be used to detect vapors of electron deficient chemicals, but require many steps to synthesize and are not selective to explosives.

Additionally, current routes for synthesis of polymetalloles use hazardous reagents and are of low efficiency. For example, poly(tetraphenyl)silole has been synthesized by Wurtz-type polycondensation, but the reaction yields are low.

### DISCLOSURE OF INVENTION

An embodiment of the present invention is a directed device and method for detecting nitroaromatic compounds using an inorganic polymer sensor, namely photoluminescent metallole copolymers. The invention also includes a method for synthesizing an inorganic polymer sensor, namely photoluminescent metallole copolymers.

### BRIEF DESCRIPTION OF THE DRAWINGS

FIG. 1 is a model of a polysilole molecule;

FIG. 2 illustrates a pair of equations for the synthesis of polygermole and polysilole according to an embodiment of the invention;

FIG. 3 illustrates a pair of equations for the synthesis of silole-germole copolymer according to an embodiment of the invention;

FIG. 4 illustrates a pair of equations for the synthesis of silole-silane alternating copolymers according to an embodiment of the invention;

FIG. 5 is a table of the absorption and fluorescence spectra observed in one embodiment of the instant invention and taken at the concentrations of 2 mg/L in THF and 10 mg/L in toluene, respectively;

FIG. 6 is a schematic energy level diagram illustrating energy-levels for polymetalloles and metallole-silane copolymers;

FIG. 7 is a graphical representation of UV-vis absorption spectra in THF (solid line) and fluorescence spectra in toluene (dotted line) for (A) poly(tetraphenyl) germole 2. (B) silole-silane copolymer 4, and (C) germole-silane copolymer 9;

FIGS. 8A and 8B illustrate a HOMO (A) and LUMO (B) of 2.5-diphenylsilole, Ph₂C₄SiH₂ from the ab initio calculations at the HF/6-31G* level;

FIG. 9 is a graphical representation of the fluorescence spectra of polysilole 1 in toluene solution (solid line) and in thin solid film (dotted line);

FIG. 10 is a graphical representation of the quenching of photoluminescence spectra of silole-silane copolymer with (A) nitrobenzene, from top 2.0×10-5 M,; 3.9×10⁻⁵ M, 7.8×10⁻⁵ M, and 11.5×10⁻⁵ M, (B) DNT, from top 1.4×10⁻⁵ M, 3.9×10⁻⁵ M, 7.8×10⁻⁵ M, and 12.4×10⁻⁵ M, (C) TNT, from top 2.1×10⁻⁵ M, 4.2×10⁻⁵ M, 8.1×10⁻⁵ M, and 12.6×10⁻⁵ M, (D) picric acid, from top 2.1×10⁻⁵ M, 4.2×10⁻⁵ M, 8.0×10⁻⁵ M, and 12.6×10⁻⁵ M;

FIGS. 11A, 11B and 11C are Stern-Volmer plots; from top polysilole 1, polygermole 2, and silole-silane copolymer 8; (picric acid), (TNT), (DNT), (nitrobenzene); the plots of fluorescence lifetime (τₒ/τ), shown as inset, are independent of added TNT;

FIG. 12 illustrates fluorescence decays of polysilole 1 for different concentrations of TNT: 0 M, 4.24×10 M, 9.09×10⁻⁵ M, 1.82×10⁻⁴ M;

FIG. 13 illustrates Stern-Volmer plots of polymers (polymer 1), (polymer 5), (polymer 4), (polymer 6), (polymer 2), and—(organic pentiptycene-derived polymer 13), for TNT;

FIG. 14 illustrates a structure of the pentiptycene-derived polymer;

FIG. 15 illustrates highest and lowest photoluminescence quenching efficiency for picric acid (purple), TNT (yellow), DNT (green), and nitrobenzene (blue) showing how the varying polymer response to analyte could be used to distinguish analytes from each other;

FIG. 16 illustrates a comparison of the photoluminescence quenching constants (from Stern-Volmer plots) of polymers 1-12 with different nitroaromatic analytes;

FIG. 17 illustrates a plot of log K vs reduction potential of analytes: (polymer 1), (polymer 2), (polymer 3), (polymer 4), (polymer 5), and—(polymer 10);

FIG. 18 illustrates a schematic diagram of electron-transfer mechanism for quenching the photoluminescence of polymetallole by analyte;

FIG. 19 illustrates an absence of quenching of photoluminescence by polysilole 1 with 4 parts per hundred of THF; and

FIG. 20 illustrates an equation for a catalytic dehyrdocoupling method for synthesizing metallole polymers according to one embodiment of the invention.

### BEST MODE FOR CARRYING OUT THE INVENTION

The instant invention is directed to the synthesis and use of inorganic polymers, namely photoluminescent metallole polymers and copolymers, for detection of nitroaromatic compounds based on photoluminescence quenching. The invention includes an inexpensive and highly efficient inorganic polymer sensor that can detect the existence of an analyte, namely nitroaromatic compounds such as picric acid (PA, 2,4,6-Trinitrophenol or C₆H₂(NO₂)₃OH), nitrobenzene (NB or C₆H₅NO₂), 2,4-dinitrotoluene (DNT or C₇H₆N₂O₄) and 2,4,6-trinitrotoluene (TNT or C₇H₅N₃O₆) in air, water, or other complex aqueous media. The invention also includes a new method for synthesizing the inorganic polymer.

Photoluminescent metallole copolymers are stable in air, water, acids, common organic solvents, and even seawater containing bioorganisms. Therefore, the inorganic polymer sensor of the instant invention includes the metallole copolymers for detection of analytes in these media. Importantly, the inorganic polymer sensor of the instant invention is insensitive to organic solvents and common environmental interferents, allowing the use of the sensor in a wide variety of environments and applications.

Metalloles are silicon (Si) or germanium (Ge)-containing metallocyclopentadienes that include one-dimensional Si—Si, Ge—Ge, or Si—Ge wires encapsulated with highly conjugated organic ring systems as side chains. Silole and germole dianions (RC)₄Si²⁻ and (RC)₄Ge²⁻, where R=Ph or Me, have been studied by X-ray crystallography and found to be extensively delocalized. Siloles and germoles are of special interest because of their unusual electronic and optical properties, and because of their possible application as electron transporting materials in devices. Polysilanes and polygermanes containing a metal-metal backbone emit in the near UV spectral region, exhibit high hole mobility, and show high nonlinear optical susceptibility, which makes them efficient photoemission candidates for a variety of optoelectronics applications. These properties arise from a σ-σ* delocalization along the M-M backbones and confinement of the conjugated electrons along the backbone.

Polymetalloles and metallole-silane copolymers are unique in having both a M-M backbone as well as an unsaturated five-membered ring system. These polymers are highly photoluminscent, and are accordingly useful as light emitting diodes (LEDs) or as chemical sensors. Characteristic features of polymetalloles and metallole-silane copolymers include a low reduction potential and a low-lying lowest unoccupied molecular orbital (LUMO) due σ*-λ* conjugation arising from the interaction between the σ* orbital of silicon or germanium and the π* orbital of the butadiene moiety of the five membered ring. In addition, the M-M backbones exhibit σ*-σ* delocalization, which further delocalizes the conjugated metallole λ electrons along the backbone. Electron delocalization in these polymers provides a means of amplification, because interaction between an analyte molecule and any position along the polymer chain is communicated throughout the delocalized chain.

Detection may be accomplished by measurement of the quenching of photoluminescence of metallole copolymers by the analyte. Sensitivity of metallole copolymers to the analytes picric acid, TNT, DNT and NB is as follows: PA>TNT>DNT>NB. A plot of log K versus the reduction potential of analytes (NB, DNT, and TNT) for each metallole copolymer yields a linear relationship, indicating that the mechanism of quenching is attributable to electron transfer from the excited metallole copolymers to the lowest unoccupied orbital of the analyte.

Excitation may be achieved with electrical or optical stimulation. If optical stimulation is used, a light source containing energy that is larger than the wavelength of luminescence emission of the polymer is preferably used. This could be achieved with, for example, a mercury lamp, a blue light emitting diode, or an ultraviolet light emitting diode.

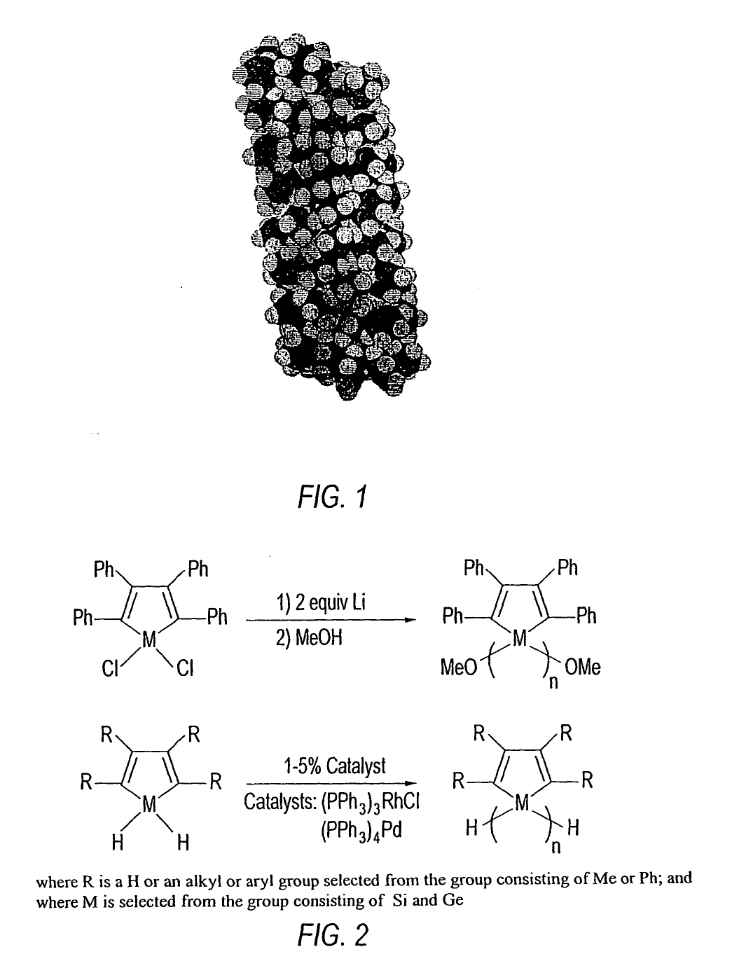

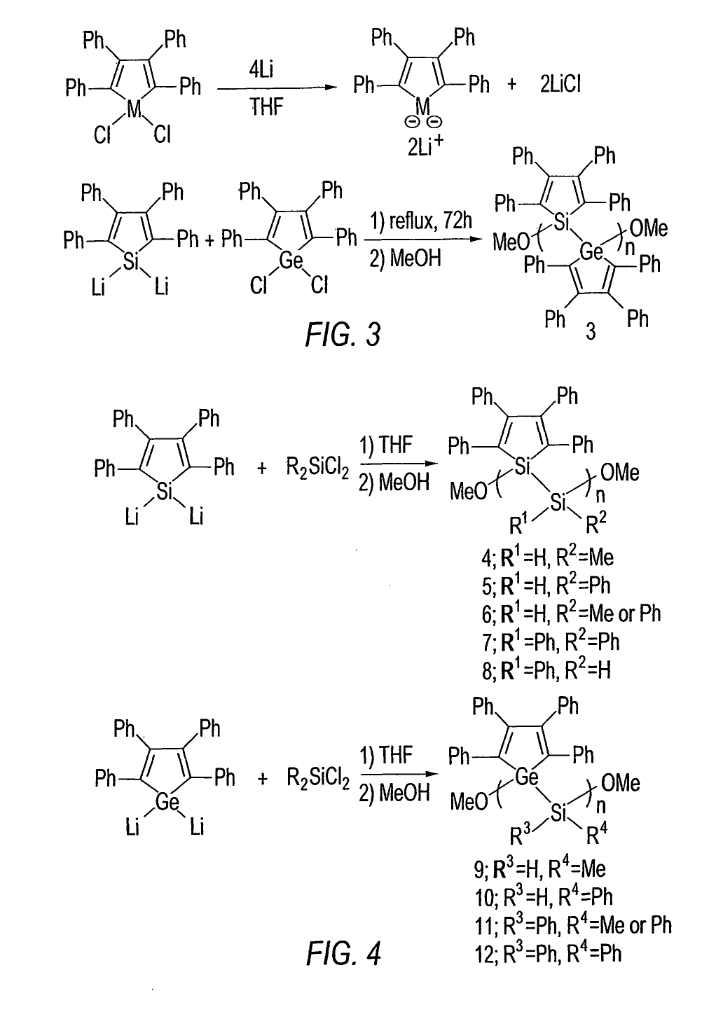

FIG. 1 illustrates a space filling model structure of polysilole 1, which features a Si—Si backbone inside a conjugated ring system of side chains closely packed to yield a helical arrangement. FIG. 2 illustrates polymers 1 and 2, FIG. 3 illustrates polymer 3, and FIG. 4 illustrates copolymers 412. A similar means of amplification is available to quantum-confined semiconductor nanocrystallites, via a three-dimensional crystalline network, where the electron and hole wave functions are delocalized throughout the nanocrystal.

A conventional method for preparing polymetalloles and metallole copolymers is Wurtz-type polycondensation. The syntheses of polygermole and polysiloles, and other copolymers are analogous to one another, as illustrated in equation 1 in FIG. 2, and employ the Wurtz-type polycondensation. However, yields from this method of synthesis are low (ca. ˜30%). Thus, Wurtz-type polycondensation is not well-suited to large-scale production.

Embodiments of the instant invention include alternative methods for synthesizing polymetalloles that use catalytic dehydrocoupling of dihydrosilotes with a catalyst as an attractive alternative to Wurtz-type polycondensation. Bis(cyclopentadienyl) complexes of Group 4 have been extensively studied and shown to catalyze the dehydrocoupling of hydrosilanes to polysilanes for the formation of Si—Si bonds. However, only the primary organosilanes react to give polysilane. Secondary and tertiary silanes give dimers or oligomers in low yield. It has been reported that the reactivity decreases dramatically with increasing substitution at the silicon atom, since reactions catalyzed by metallocenes are typically very sensitive to steric effects. Mechanisms for dehydrogenative coupling of silanes have also been extensively investigated, which involves σ-bond metathesis.

Embodiments of the instant invention include catalytic dehydrocoupling of dihydrosiloles and dihydrogermoles with a catalyst. In one embodiment, the invention includes catalytic dehydrocoupling polycondensation of dihydro(tetraphenyl)silole or dihydro(tetraphenyl)germole with 1-5 mol % of Wilkinson's catalyst, Rh(PPh₃)₃Cl, or Pd(PPh₃)₄, as illustrated in FIG. 2, or 0.1-0.5 mol % of H₂PtCl₆ xH₂O in conjuction with 2-5 equivalents of allylamine, as illustrated in FIG. 20. The latter reactions produce the respective polysilole or polygermole in high yield (ca. 80-90%). By ¹H NMR spectroscopy, the monomer, dihydrometallole, was completely consumed in the reaction. Molecular weights (M_{w}) of 4000˜6000 are obtained, similar to those obtained by the Wurtz-type polycondensation (ca. ˜30%).

Turning now to FIG. 3, silole-germole alternating copolymer 3, in which every other silicon or germanium atom in the polymer chain is also part of a silole or germole ring, was synthesized from the coupling of dichloro(tetraphenyl)germole and dilithio(tetraphenyl)silole. The latter is obtained in 39% yield from dichlorotetraphenylsilole by reduction with lithium, as illustrated in the equation of FIG. 3. The molecular weight of the silole-germole copolymer, M_{w}=5.5×10³, Mₙ=5.0×10³ determined by SEC (size exclusion chromatography) with polystyrene standards, is similar to that of polysiloles or polygermoles. All of the polymetalloles are extended oligomers with a degree of polymerization of about 10 to 16, rather than a true high M_{w} polymer; however, they can be cast into a thin film from solution and show polymer-like properties.

Also illustrated in FIG. 4 are silole-silane alternating copolymers 4, 5, 6, 7, 8, which were also prepared from coupling of the silole dianion (Ph₄C₄Si)Li₂ with the corresponding silanes. Germole-silane alternation copolymers 9, 10, 11, 12 were also synthesized from the coupling of the germole dianion (Ph4C₄Ge)Li₂ with the corresponding silanes, as illustrated in FIG. 4. These reactions generally employ reflux conditions in tetrahydrofuran under an argon atmosphere for about 72 hours. Some silole-silane copolymers have been synthesized previously and shown to be electroluminescent. Metallole-silane copolymers were developed so that they could be easily functionalized along the backbone by hydrosilation. The molecular weight of metallole-silane copolymers, M_{w}=4.1×10³˜6.2×10³, Mₙ=4.1×10³˜5.4×10³ determined by SEC, is similar to that of the polymetalloles.

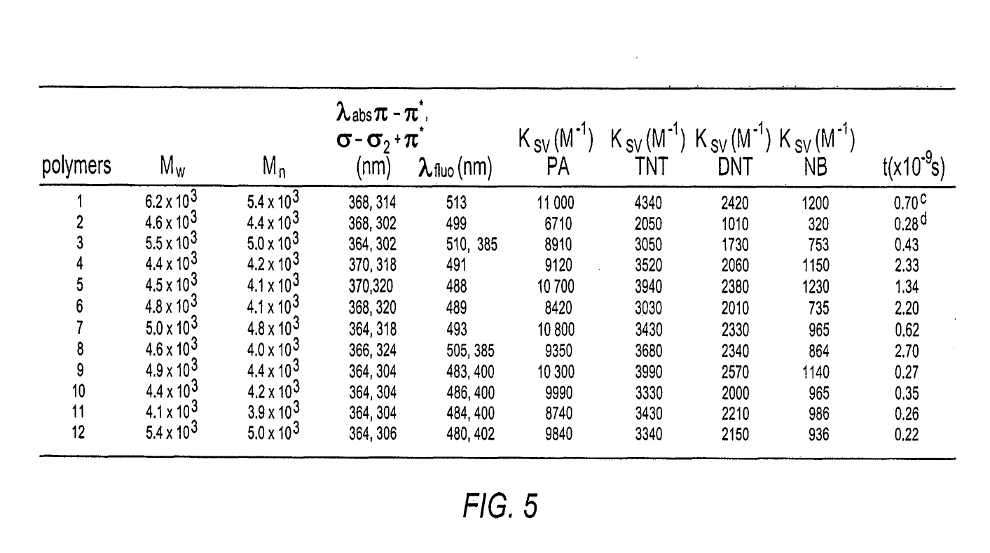

The molecular weights and polydisperity indices (PDI) of polymers 1-12 (FIG. 4) determined by gel permeation chromatography (GPC) are illustrated in Table 1 of FIG. 5.

Absorption and Fluorescence

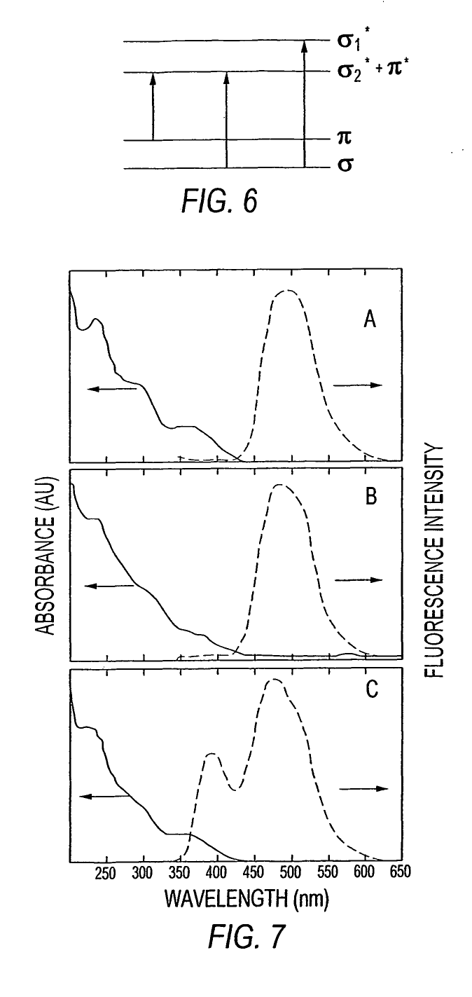

The UV-vis absorption and fluorescence spectral data for polymers 1-12 are also illustrated in Table 1 of FIG. 5. The poly(tetraphenyl)metalloles 1-3 and tetraphenylmetallole-silane copolymers 4-12 exhibit three absorption bands, which are ascribed to the π-π* transition in the metallole ring and the σ−(σ*+π*) and σ-σ* transitions in the M-M backbone. FIG. 6 illustrates a schematic energy-level diagram for polymetalloles and metallole-silane copolymers.

UV-vis absorption in THF (solid line) and fluorescence spectra in toluene (dotted line) for poly(tetraphenygermole) 2, silole-silane copolymer 4 and germole-silane copolymer 9 are shown in FIG. 7. Absorptions at a wavelength of about 370 nm for the poly(tetraphenylmetallole)s 1-3 and tetraphenylmetallole-silane copolymers 4-12 are ascribed to the metallole π-π* transition of the metallole moiety, which are about 89 to 95 nm red-shifted relative to that of oligo[1,1-(2,3,4,5-tetramethylsilole)] (λₘₐₓ=275 nm) and are about 75 to 81 nm red-shifted relative to that of oligo[1,1-(2,5-dimethyl-3,4-diphenylsilole)] (λₘₐₓ=289 nm). These red shifts are attributed to an increasing main chain length and partial conjugation of the phenyl groups to the silole ring.

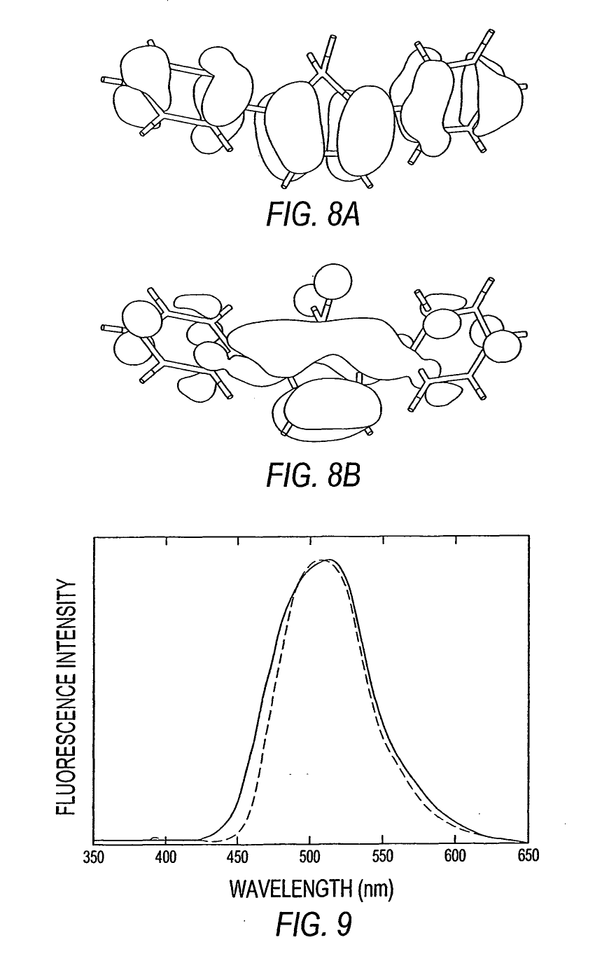

FIG. 8 shows the HOMO (A) and LUMO (B) of 2,5-diphenylsilole, Ph2C4SiH2, from the ab initio calculations at the HF/6-31G* level. Phenyl substituents at the 2,5 metallole ring positions may n-conjugate with the metallole ring LUMO. Second absorptions at wavelengths of 304 to 320 nm for the poly(tetraphenylmetallole)s 2-3 and tetraphenylmetallole-silane copolymers 4-12 are assigned to the σ−(σ₂*+π*) transition, which parallels that of the poly(tetraphenyl)silole 1.

Polymetallole 1-2 and silole-silane copolymers 47 exhibit one emission band (λₘₐₓ, 486 to 513 nm) when excited at 340 nm, whereas the others exhibit two emission bands with λₘₐₓ of 480-510 nm and 385-402 nm. The ratios of the two emission intensities are not concentration dependent, which indicates that the transition does not derive from an excimer. Emission peaks for germole-silane copolymers 9-12 are only 2 to 33 nm blue-shifted compared to the other polymers. FIG. 9 shows fluorescence spectra of the poly(tetraphenyl)silole in toluene solution (solid line) and in the solid state (dotted line). The bandwidth of the emission spectrum in solution is slightly larger than in the solid state. There is no shift in the maximum of the emission wavelength. This suggests that the polysilole exhibits neither π-stacking of polymer chains nor excimer formation.

The angles of C-M-C of dihydro(tetraphenyl)silole and dihydro(tetraphenyl)germole are 93.11° on C—Si—C and 89.76° on C—Ge—C, respectively. Polymerization might take place, since the tetraphenylmetalloles have small angles at C-M-C in the metallocyclopentadiene ring, which results in less steric hindrance at the metal center. In addition, the bulky phenyl groups of silole might prevent the formation of cyclic hexamer, which is often problematic in polysilane syntheses. Cyclic polymetallole product formation was not observed.

Fluorescence Quenching with Nitroaromatic Analytes

The method of detection of the instant invention includes using a chemical sensor, namely a variety of photoluminscent copolymers having a metalloid-metalloid backbone such as Si—Si, Si—Ge, or Ge—Ge. While polymetalloles in various forms may be used to detect analytes, one embodiment includes casting a thin film of the copolymers is employed in detecting the analyte, e.g., picric acid, DNT, TNT and nitrobenzene. Detection is achieved by measuring the quenching of the photoluminescence of the copolymer by the analyte. Accordingly, the instant invention contemplates use of the polymetallole polymers and copolymers in any form susceptible to measurement of photoluminescence quenching. For example, since it is possible to measure fluorescence of solutions, other embodiments of the instant method of detection may optionally include a polymetallole in solution phase, where powdered bulk polymer is dissolved in solution. Yet another embodiment includes producing a colloid of the polymer, which is a liquid solution with the polymer precipitated and suspended as nanoparticles.

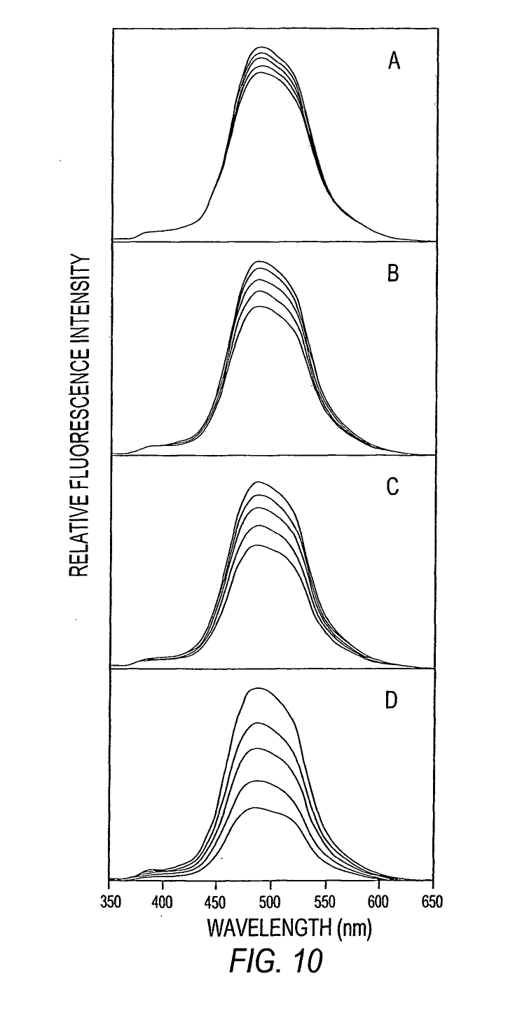

The detection method involves measurement of the quenching of photoluminescence of the polymetalloles 1-3 and metallole-silane copolymers 4-12 by the analyte, such as a toluene solution (using a Perkin-Elmer LS 50B fluorescence spectrometer, 340 nm excitation wavelength). For example, turning now to FIG. 10, when used to detect TNT, fluorescence spectra of a toluene solution of the metallole copolymers were obtained upon successive addition of aliquots of TNT. Photoluminescence quenching of the polymers 1-12 in toluene solutions were also measured with nitrobenzene, DNT, TNT and nitrobenzene. The relative efficiency of photoluminescence quenching of metallole copolymers is unique for TNT, DNT, and nitrobenzene, respectively, as indicated in FIG. 10 by the values of K determined from the slopes of the steady-state Stern-Volmer plots. FIG. 10 demonstrates that each copolymer has a unique ratio of quenching efficiency to the corresponding analyte.

The purity of the TNT sample was found to be important to obtain reproducible results. It was synthesized by nitration of dinitrotoluene and recrystallized twice from methanol. A third recrystallization produces the same results as the twice-recrystallized material. When the quenching experiment was undertaken without recrystallization of TNT, higher (ca. 10×) quenching percentages are obtained. Presumably, impurities with higher quenching efficiencies are present in crude TNT.

The Stem-Volmer equation, which is (I_{O}/I)−1=K_{SV}[A], is used to quantify the differences in quenching efficiency for various analytes. In this equation, I_{O} is the initial fluorescence intensity without analyte, and I is the fluorescence intensity with added analyte of concentration [A], and K_{SV} is the Stern-Volmer constant.

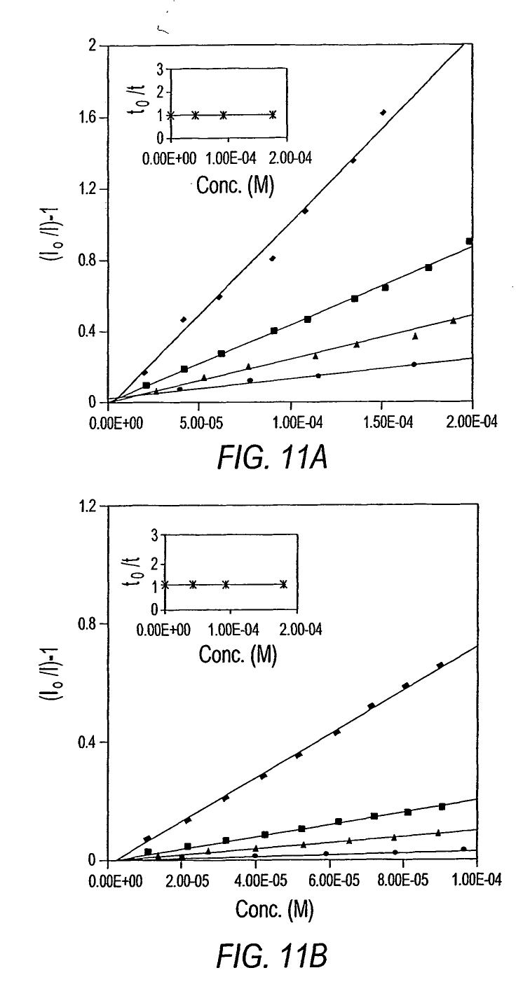

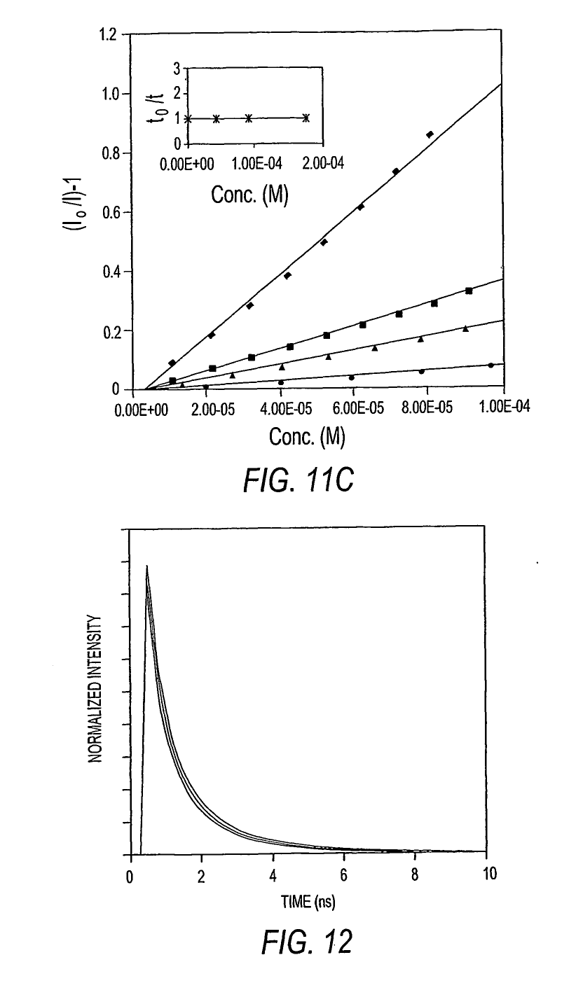

FIG. 11 shows the Stern-Volmer plots of polysilole 1, polygermole 2, and silole-silane copolymer 8 for each analyte. A linear Stern-Volmer relationship was observed in all cases, but the Stem-Volmer plot for picric acid exhibits an exponential dependence when its concentration is higher than 1.0×10⁻⁴ M. A linear Stern-Volmer relationship may be observed if either static or dynamic quenching process is dominant. Thus, in the case of higher concentrations of picric acid, the two processes may be competitive, which results in a nonlinear Stern-Volmer relationship. This could also arise from aggregation of analyte with chromophore.

Photoluminescence may arise from either a static process, by the quenching of a bound complex, or a dynamic process, by collisionally quenching the excited state. For the former case, Ksv is an association constant due to the analyte-preassociated receptor sites. Thus, the collision rate of the analyte is not involved in static quenching and the fluorescence lifetime is invariant with the concentration of analyte. With dynamic quenching, the fluorescence lifetime should diminish as quencher is added.

A single “mean” characteristic lifetime (,c) for polymetalloles and metallole-silane copolymers 1-12 has been measured and summarized in Table 1 of FIG. 5. Luminescence decays were not single-exponential in all cases. Three lifetimes were needed to provide an acceptable fit over the first few nanoseconds. The amplitudes of the three components were of comparable importance (the solvent blank made no contribution). These features suggest that the complete description of the fluorescence is actually a continuous distribution of decay rates from a heterogeneous collection of chromophore sites. Because the oligomers span a size distribution, this behavior is not surprising. The mean lifetime parameter reported is an average of the three lifetimes determined by the fitting procedure, weighted by their relative amplitudes. This is the appropriate average for comparison with the “amount” of light emitted by different samples under different quenching conditions, as has been-treated in the literature. Given this heterogeneity, possible long-lived luminescence that might be particularly vulnerable to quenching has been a concern. However, measurements with a separate nanosecond laser system confirmed that there were no longer-lived processes other than those captured by the time-correlated photon counting measurement and incorporated into Table 1 of FIG. 5.

It is notable that polysilole 1 and silole-silane copolymers 4-8 have about 3 to 11 times longer fluorescence lifetimes than polygermole 2 and germole-silane copolymers 9-12. Fluorescence lifetimes in the thin films (solid state) for polysilole 1 and polygermole 2 are 2.5 and 4.2 times longer than in toluene solution, respectively. The fluorescence lifetimes as a function of TNT concentration were also measured and are shown in the inset of FIG. 11 for polymers 1, 2, and 8. No change of mean lifetime was observed by adding TNT, indicating that the static quenching process is dominant for polymetalloles and metallole-silane copolymers 1-12 (FIG. 12). Some issues with such analyses have been discussed in the literature. This result suggests that the polymetallole might act as a receptor and a TNT molecule would intercalate between phenyl substituents of the metallole moieties (FIG. 1).

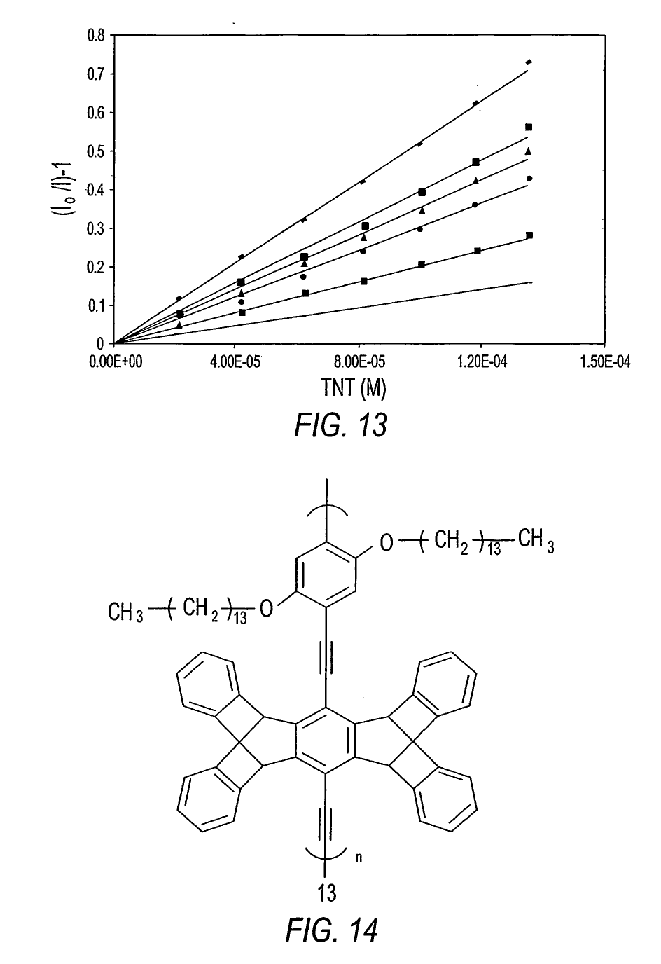

For chemosensor applications, it is useful to have sensors with varied responses. Each of the 12 polymers exhibits a different ratio of the photoluminescence quenching for picric acid, TNT, DNT, and nitrobenzene and a different response with the same analyte. The use of sensor arrays is inspired by the performance of the olfactory system to specify an analyte. FIG. 13 displays the Stem-Volmer plots of polymers 1, 2, 4, 5, and 6 for TNT, indicating that the range of photoluminescence quenching efficiency for TNT is between 2.05×10³ and 4.34×10³ M⁻¹. The relative efficiencies of photoluminescence quenching of poly(tetraphenylmetallole)s 1-3 and tetraphenyl-metallole-silane copolymers 4-12 were obtained for picric acid, TNT, DNT, and nitrobenzene, as indicated by the values of Ksv determined from the slopes of the steady-state Stern-Volmer plots and summarized in Table 1 of FIG. 5. Polymer 13, which is illustrated in FIG. 14, is an organic pentiptycene-derived polymer for comparison. The metallole copolymers are more sensitive to TNT than the organic pentiptycene-derived polymers in toluene solution. For example, polysilole 1 (4.34×10³ M⁻¹) has about a 370% better quenching efficiency with TNT than organic pentiptycene-derived polymer (1.17×103 M⁻¹).

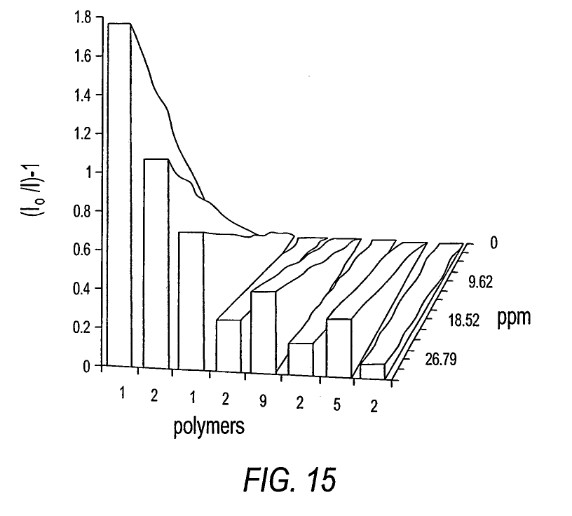

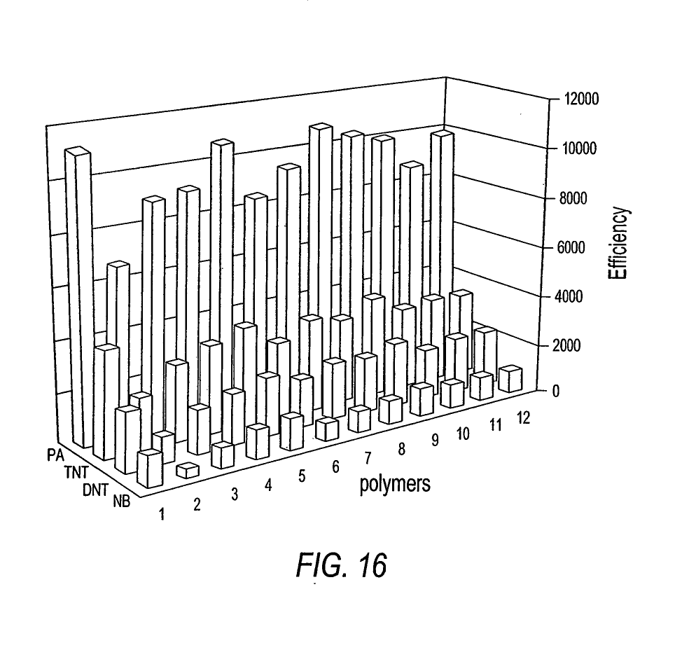

The trend in Stern-Volmer constants usually reflects an enhanced charge-transfer interaction from metallole polymer to analyte. For example, the relative efficiency of photoluminescence quenching of polysilole 1 is about 9.2:3.6:2.0:1.0 for picric acid, TNT, DNT, and nitrobenzene, respectively. Although polysilole 1 shows best photoluminescence quenching efficiency for picric acid and TNT, polymer 9 and 5 exhibit best quenching efficiency for DNT and nitrobenzene, respectively. (FIG. 15) Polygermole 2 has the lowest quenching efficiency for all analytes. Since the polymers 1-12 have similar molecular weights, the range of quenching efficiencies with the same analyte would be expected to be small. Polysilole 1 (11.0×10³ M⁻¹ and 4.34×10³ M⁻¹) exhibits 164% and 212% better quenching efficiency than polygermnole 2 (6.71×103 M⁻¹ and 2.05×10³ M⁻¹) with picric acid and TNT, respectively. Polymer 9 (2.57×10³ M⁻¹) has 253% better quenching efficiency than polymer 2 (1.01×10³ M⁻¹) with DNT. Polymer 5 (1.23×10⁻³ M⁻¹) has 385% better quenching efficiency than metallole polymer 2 (0.32×10³ M⁻¹) with nitrobenzene. FIG. 16 illustrates how an analyte might be specified using an array of multi-sensors.

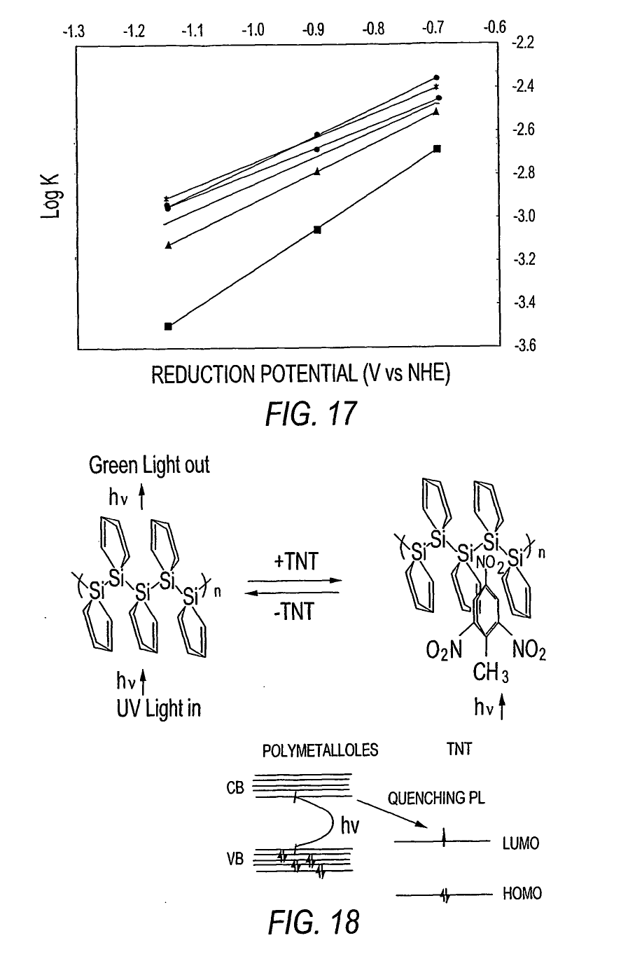

FIG. 17 shows a plot of log Ksv vs. reduction potential of analytes. All metallole polymers exhibit a linear relationship, even though they have different ratios of photoluminescence quenching efficiency to analytes. This result indicates that the mechanism of photoluminescence quenching is primarily attributable to electron transfer from the excited metallole polymers to the LUMO of the analyte. Because the reduction potential of TNT (−0.7 V vs NHE) is less negative than that of either DNT (−0.9 V vs NHE) or nitrobenzene (−1.15 V vs NHE), it is detected with highest sensitivity. A schematic diagram of the electron-transfer mechanism for the quenching of photoluminescence of the metallole polymers with analyte is shown in FIG. 18. Optical excitation produces an electron-hole pair, which is delocalized through the metallole copolymers. When an electron deficient molecule, such as TNT is present, electron-transfer quenching occurs from the excited metallole copolymer to the LUMO of the analyte. The observed dependence of Ksv on analyte reduction potential suggests that for the static quenching mechanism, the polymer-quencher complex luminescence intensity depends on the electron acceptor ability of the quencher. An alternative explanation would be that the formation constant (Ksv) of the polymer-quencher complex is dominated by a charge-transfer interaction between polymer and quencher and that the formation constant increases with quencher electron acceptor ability.

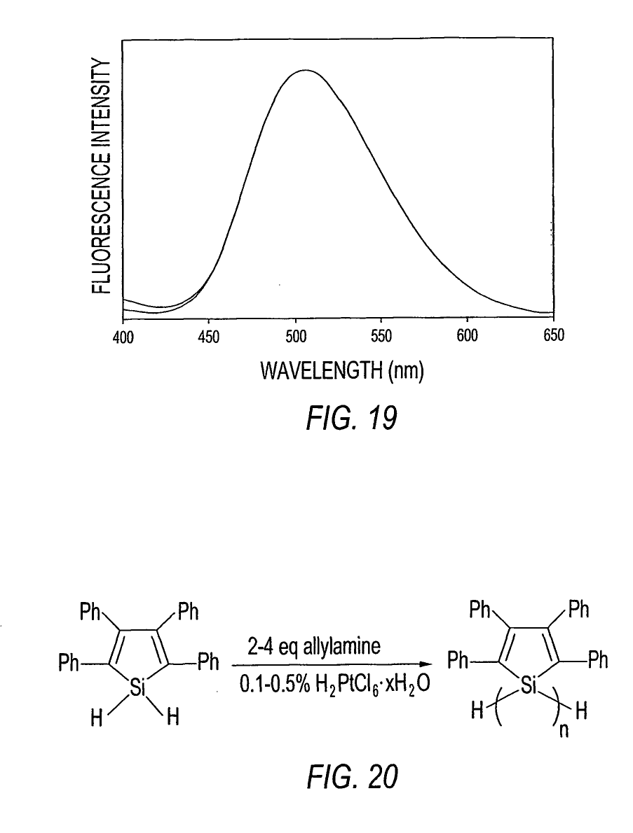

An important aspect of the metallole copolymers is their relative insensitivity to common interferents. Control experiments using both solutions and thin films of metallole copolymers (deposited on glass substrates) with air displayed no change in the photoluminescence spectrum. Similarly, exposure of metallole copolymers both as solutions and thin films to organic solvents such as toluene, THF, and methanol or the aqueous inorganic acids H₂SO₄ and HF produced no significant decrease in photoluminescence intensity. FIG. 19 shows that the photoluminescence spectra of polysilole 1 in toluene solution display no quenching of fluorescence with 4 parts per hundred of THF. The ratio of quenching efficiency of polysilole 1 with TNT vs benzoquinone is much greater than that of polymer 13. The Ksv value of 4.34×10³ M⁻¹ of polysilole 1 for TNT is 640% greater than that for benzoquinone (Ksv=674 M⁻¹). The organic polymer 13, however, only exhibits a slightly better quenching efficiency for TNT (Ksv=1.17×10³ M⁻¹) (ca. 120%) compared to that (Ksv=998 M⁻¹) for benzoquinone. This result indicates that polysilole 1 exhibits less response to interferences and greater response to nitroaromatic compounds compared to the pentiptycene-derived polymer 13.

Statistical Estimates of Detection Limit from Extrapolation of Stern-Volmer Quenching Data:

From Stem-Volmer Quenching Data:

Of log(Io/I)−1 vs [TNT] in ppb.

This corresponds to an extrapolated detection limit of ˜1.5 ppt for instant detection with our fluorescence spectrometer at the 95% confidence limit. Of course, this is for solution data and with a spectrometer, which is not optimized for detection at a single wavelength.

### EXAMPLE

All synthetic manipulations were carried out under an atmosphere of dry dinitrogen gas using standard vacuum-line Schlenk techniques. All solvents were degassed and purified prior to use according to standard literature methods: diethyl ether, hexanes, tetrahydrofuran, and toluene purchased from Aldrich Chemical Co. Inc. were distilled from sodium/benzophenone ketal. Spectroscopic grade of toluene from Fisher Scientific was used for the fluorescent measurement. NMR grade deuteriochloroform was stored over 4 A molecular sieves. All other reagents (Aldrich, Gelest) were used as received or distilled prior to use. NMR data were collected with Varian Unity 300, 400, or 500 MHz spectrometers (300.1 MHz for ¹H NMR, 75.5 MHz for ¹³C NMR and 99.2 MHz for ²⁹Si NMR) and all NMR chemical shifts are reported in parts per million (δ ppm); downfield shifts are reported as positive values from tetramethylsilane (TMS) as standard at 0.00 ppm. The ¹H and ¹³C chemical shifts are reported relative to CHCl₃ (δ 77.0 ppm) as an internal standard, and the ²⁹Si chemical shifts are reported relative to an external TMS standard.

NMR spectra were recorded using samples dissolved in CDCl₃, unless otherwise stated, on the following instrumentation. ¹³C NMR were recorded as proton decoupled spectra, and ²⁹Si NMR were recorded using an inverse gate pulse sequence with a relaxation delay of 30 seconds. The molecular weight was measured by gel permeation chromatography using a Waters Associates Model 6000A liquid chromatograph equipped with three American Polymer Standards Corp. Ultrastyragel columns in series with porosity indices of 10³, 10⁴, and 10⁵ Å, using freshly distilled THF as eluent.

The polymer was detected with a Waters Model 440 ultraviolet absorbance detector at a wavelength of 254 nm, and the data were manipulated using a Waters Model 745 data module. Molecular weight was determined relative to calibration from polystyrene standards. Fluorescence emission and excitation spectra were recorded on a Perkin-Elmer Luminescence Spectrometer LS 50B. Monomers, 1,1-dichloro-2,3,4,5-tetraphenylsilole, 1,1-dichloro-2,3,4,5-tetraphenylgermole, 1,1-dilithio-2,3,4,5-tetraphenylsilole, and 1,1-dilithio-2,3,4,5-tetraphenylgermole were synthesized by following the procedures described in the literature. All reactions were performed under Ar atmosphere.

Polymetalloles 5,6, and 7 were synthesized by following the procedures described in the literature.

Preparation of silole-silane copolymers, (silole-SiR¹R²),: Stirring of 1,1-dichloro-2,3,4,5-tetraphenylsilole (5.0 g, 11.0 mmol) with lithium (0.9 g, 129.7 mmol) in THF (120 mL) for 8 h at room temperature gave a dark yellow solution of silole dianion. After removal of excess lithium, 1 mol equiv of corresponding silanes, R¹R²SiCl₂(11.0 mmol) was added slowly to a solution of tetraphenylsilole dianion, and stirred at room temperature for 2 hours. The resulting mixture was refluxed for 3 days. The reaction mixture was cooled to room temperature and quenched with methanol. Then the volatiles were removed under reduced pressure. THF (20 mL) was added to the residue and polymer was precipitated by slow addition of the solution into 700 mL of methanol. The third cycle of dissolving-precipitation followed by freeze-drying gave the polymer as yellow powder.

For (silole)ₙ(SiMeH)ₘ(SiPhH)ₒ, each 5.5 mmol of SiMeHCl₂ and SiPhHCl₂ were slowly added into a THF solution of silole dianion. In case of (silole-SiH₂)ₘ, after addition of the xylene solution of SiH₂Cl₂ (11.0 mmol), the resulting mixture was stirred for 3 days at room temperature instead of refluxing.

Selected data for (silole-SiMeH)ₙ, 1; Yield=2.10 g (44.5%); ¹H NMR (300.134 MHz, CDCl₃): δ=−0.88-0.60 (br. 3H, Me), 3.06-4.89 (br. 1H, SiH, 6.16-7.45 (br. 20H, Ph); ¹³C{H} NMR (75.469 MHz, CDCl₃): δ=0.61-1.69 (br. Me), 123.87-131.75, 137.84-145.42, 153.07-156.73 (br. m, Ph); ²⁹Si NMR (71.548 MHz, inversed gated decoupling, CDCl₃): δ=−29.22 (br. silole), −66.61 (br. SiMeH). GPC: Mw=4400, Mw/Mn=1.04. Fluorescence (conc.=10 mg/L); λₑₘ=492 nm at λₑₓ=340 nm.

Selected data for (silole-SiPhH)ₙ, 2; Yield=2.00 g (37.0%); ¹H NMR (300.134 MHz, CDCl₃): δ=3.00-4.00 (br. 1H, SiH), 6.02-7.97 (br. 20H, Ph); ¹³C{H} NMR (75.469 MHz, CDCl₃): δ=123.64-143.98, 152.60-157.59 (br. m, Ph); ²⁹Si NMR (71.548 MHz, inversed gated decoupling, CDCl₃): δ=−37.51 (br. silole), −71.61 (br. SiPhH). GPC: Mw=4500, Mw/Mn=1.09, determined by SEC with polystyrene standards; Fluorescence (conc.=10 mg/L); λₑₘ=487 nm at λₑₓ=340 nm.

Selected data for (silole)ₙ(SiMeH)_{0.5n}(SiPhH)_{0.5n}, 3; Yield=2.10 g (41.5%); ¹H NMR (300.134 MHz, CDCl₃): δ=−0.67-0.40 (br. 3H, Me), 3.08-4.98 (br. 2H, SiH), 6.00-7.82 (br. 55H, Ph); ¹³C{H} NMR (75.469 MHz, CDCl₃): δ=−0.85-1.76 (br. Me), 122.06-147.25, 153.11-157.26 (br. m, Ph); ²⁹Si NMR (71.548 MHz, inversed gated decoupling, CDCl₃): δ=−28.61 (br. silole), −59.88 (br. SiMeH and SiPhH). GPC: Mw=4800, Mw/Mn=1.16, determined by SEC with polystyrene standards; Fluorescence (conc.=10 mg/L); λₑₘ=490 nm at λₑₓ=340 nm.

Selected data for (silole-SiH₂)ₙ, 4; Yield=2.05 g (44.9%); ¹H NMR (300.134 MHz, CDCl₃): δ=3.00-4.96 (br. 2H, SiH₂), 6.12-7.72 (br. 20H, Ph); ¹³C{H} NMR (75.469 MHz, CDCl₃): δ=122.08-132.78, 136.92-146.25, 152.81-160.07 (br. m, Ph); ²⁹Si NMR (71.548 MHz, inversed gated decoupling, CDCl₃): δ=−30.95 (br. silole), −51.33 (br. SiH₂). ratio of n:m=1.00:0.80; GPC: Mw=4600, Mw/Mn=1.14, determined by SEC with polystyrene standards; Fluorescence (conc.=10 mg/L); λₑₘ=499 nm at λₑₓ=340 nm.

Selected data for (silole-SiPh₂)ₙ, 5; Yield=2.93 g (47.0%); ¹H NMR (300.134 MHz, CDCl₃): δ=6.14-7.82 (br. 20H, Ph); ¹³C{H} NMR (75.469 MHz, CDCl₃): δ=122.08-146.25 (br. m, Ph), 152.81-160.07 (silole ring); GPC: Mw=5248, Mw/Mn=1.05, determined by SEC with polystyrene standards; Fluorescence (conc.=10 mg/L); λₑₘ=492 nm at λₑₓ =340 nm.

Preparation of germole-silane copolymers, (germole-SiR¹R²),:

The procedure for synthesizing all germole-silane copolymers was similar to that for silole-silane copolymers. For (germole)ₙ(SiMeH)_{0.5n}(SiPhH)_{0.5n}, each 5.0 mmol of SiMeHCl₂ and SiPhHCl₂ were added slowly into a THF solution of germole dianion. The resulting mixture was stirred for 3 days at room temperature.

Selected data for (germole-SiMeH)ₙ, 6; Yield=2.03 g (43%); ¹H NMR (300.134 MHz, CDCl₃): δ=−0.21-0.45 (br. 2.4H, Me), 5.14-5.40 (br. 0.8H, SiH), 6.53-7.54 (br. 20H, Ph); ¹³C{H} NMR (75.469 MHz, CDCl₃): δ=−9.70-8.15 (br. Me), 125.29-130.94, 139.08-148.12, 151.29-152.88 (br. m, Ph); ²⁹Si NMR (71.548 MHz, inversed gated decoupling, CDCl₃): δ=−50.40 (br. SiMeH); GPC: Mw=4900, Mw/Mn=1.12, determined by SEC with polystyrene standards; UV (conc.=10 mg/L); 5abs=296, 368 nm; Fluorescence (conc.=10 mg/L); λₑₘ=401, 481 nm at λₑₓ=340 nm.

Selected data for (germole-SiPhH)ₙ, 7; Yield=2.13 g (40%); ¹H NMR (300.134 MHz, CDCl₃): δ=4.71 (br. 1.0H, SiH), 6.30-7.60 (br. 25H, Ph); ¹³C{H} NMR (75.469 MHz, CDCl₃): δ=125.50-144.50, 151.50-153.00 (br. m, Ph); ²⁹Si NMR (71.548 MHz, inversed gated decoupling, CDCl₃): δ=−56.81 (br. SiPhH).; GPC: Mw=4400, Mw/Mn=1.06, determined by SEC with polystyrene standards; UV (conc.=10 mg/L); λ_{abs}=294, 362 nm; Fluorescence (conc.=10 mg/L); λₑₘ=401, 486 nm at λₑₓ=340 nm.

Selected data for (germole)ₙ(SiMeH)_{0.5n}(SiPhH)_{0.5n}, 8; Yield=2.01 g(40%); ¹H NMR (300.134 MHz, CDCl₃): δ=−0.04-0.42 (br. 3H, Me), 4.94 (br. 2H, SiH), 6.33-7.66 (br. 25H, Ph); ¹³C{H} NMR (75.469 MHz, CDCl₃): δ=124.31-130.66, 138.43-152.54 (br. m, Ph); ²⁹Si NMR (71.548 MHz, inversed gated decoupling, CDCl₃): δ=−63.01 (br. SiMeH and SiPhH): 0.71; GPC: Mw=4100, Mw/Mn=1.06, determined by SEC with polystyrene standards; UV (conc.=10 mg/L); λ_{abs}=290, 364 nm; Fluorescence (conc.=10 mg/L); λₑₘ=399, 483 nm at λₑₓ=340 nm.

Selected data for (germole-SiPh₂)ₙ, 9;Yield=3.23 g (48%); ¹H NMR (300.134 MHz, CDCl₃): δ=6.21-7.68 (br. 30H, Ph); ¹³C{H} NMR (75.469 MHz, CDCl₃): δ=125.15-141.40 (br. m, Ph), 151.12-153.99 (germole ring carbon); GPC: Mw=5377, Mw/Mn=1.09, determined by SEC with polystyrene standards; UV (conc.=10 mg/L); λ_{abs}=298, 366 nm; Fluorescence (conc.=10 mg/L); λₑₘ=400, 480 nm at λₑₓ=340 nm.

Preparations for other metallole-silane and metallole-germane copolymers such as tetraalkylmetallole -silane copolymers and tetraarylmetallole-germane copolymers can be prepared by the above method described.

Preparation of Poly(tetraphenyl)silole and Poly(tetraphenyl)germole by Catalytic Dehydrocoupling—Preparation of polymetallole: 1,1-dihydro-2,3,4,5-tetraphenylsilole or germole were prepared from the reduction of 1,1-dichloro-2,3,4,5-tetraphenylsilole or germole with 1 mol equiv of LiAlH₄. Reaction conditions for preparing the polygermole are the same as those for polysilole. 1,1-dihydro-2,3,4,5-tetraphenylsilole (1.0 g, 2.59 mmol) and 1-5 mol % of RhCl(PPh₃)₃ or Pd(PPh₃)₄ in toluene (10 mL) were placed under an Ar atmosphere and degassed through 3 freeze-pump-thaw cycles. The reaction mixture was vigorously refluxed for 72 h. The solution was passed rapidly through a Florisil column and evaporated to dryness under Ar atmosphere. 1 mL of THF was added to the reaction mixture and the resulting solution was then poured into 10 mL of methanol. Poly(tetraphenyl)silole, 1, was obtained as a pale yellow powder after the third cycle of dissolving-precipitation followed by freeze-drying. An alternative method for poly(tetraphenyl)silole preparation is as follows. 1,1-dihydro-2,3,4,5-tetraphenylsilole (1.0 g, 2.59 mmol) and 0.1-0.5 mol % H₂PtCl₆ xH₂O and 2-5 mol equivalents of allylamine in toluene (10 mL) were vigorously refluxed for 24 hours. The solution was passed through a sintered glass frit and evaporated to dryness under an Ar atmosphere. Three dissolving-precipitation cycles with THF and methanol were performed as stated above to obtain 1. The molecular weights of polymers were obtained by GPC. 1,1-dihydro-2,3,4,5-tetraphenylsilole with RhCl(PPh₃)₃, 1: isolated yield=0.81 g, 82%, Mw=4355, Mw/Mn=1.02, determined by SEC with polystyrene standards; 1,1-dihydro-2,3,4,5-tetraphenylsilole with Pd(PPh₃)₄, 1: 0.84 g, 85%, M_{w}=5638, M_{w}/Mₙ=1.10). 1,1-dihydro-2,3,4,5-tetraphenylgermole with RhCl(PPh₃)₃, poly(tetraphenyl)germole: 0.80 g, 81%, M_{w}=3936, M_{w}/Mₙ=1.01; 1,1-dihydro-2,3,4,5-tetraphenylgermole with Pd(PPh₃)₄, poly(tetraphenyl)germole: 0.81 g, 82%, M_{w}=4221, M_{w}/Mₙ=1.02) ¹H NMR (300.133 MHz, CDCl₃): δ=6.30-7.90 (br, m, Ph); ¹³C{H} NMR (75.403 MHz, CDCl₃ (δ=77.00)): δ=124-130 (br, m, Ph), 131-139 (germole carbons). If less vigorous reflux conditions are used, with the RhCl(PPh₃)₃ and Pd(PPh₃)₄ catalysts, then corresponding dimers form along with lesser amounts of polymer. The dimer is less soluble and crystallizes from toluene.

While various embodiments of the present invention have been shown and described, it should be understood that modifications, substitutions, and alternatives are apparent to one of ordinary skill in the art. Such modifications, substitutions, and alternatives can be made without departing from the spirit and scope of the invention, which should be determined from the appended claims.

Various features of the invention are set forth in the appended claims.

1. A dehydrocoupling polycondensation method for synthesizing polymetalloles including:
obtaining a dihydrometallole that includes silicon or germanium atoms;
designating a reducing agent for preparation of dihydrometallole monomer;
measuring a predetermined molar percentage of said reducing agent corresponding to a molar amount of said dihydrometallole;
selecting a catalyst; and
reacting said catalyst with said dihydrometallole to obtain a polymetallole.

2. The method of claim 1 wherein said step of obtaining a dihydrometallole comprises reducing a dichlorometallole and subsequently catalytically dehydrocoupling the reduced dichlorometallole to yield a polymer.

3. The method of claim 1 wherein said step of obtaining a dihydrometallole comprises adding dichlorosilane to a solution of lithium and diphenylacetylene and subsequently catalytically dehydrocoupling a product to yield a polymer.

4. The method of claim 1 wherein said obtained dihydrometallole is 1,1-dihydro-2,3,4,5 -tetraphenylsilole.

5. The method of claim 1 wherein said obtained dihydrometallole is 1,1-dihydro-2,3,4,5-tetraphenylgermole.

6. The method of claim 1 wherein said reducing agent is designated as LiAlH₄.

7. The method of claim 1 wherein said catalyst is Wilkinson's catalyst, which is Rh(PPh₃)₃Cl.

8. The method of claim 7 further comprising selecting said predetermined molar percentage of said Wilkinson's catalyst to be between 1 and 5 mol %.

9. The method of claim 1 wherein said catalyst is selected to be Pd(PPh₃)₄.

10. The method of claim 9 further comprising selecting said predetermined molar percentage of Pd(PPh₃)₄ to be between 1 and 5 mol %.

11. The method of claim 1 wherein said catalyst is selected to a combination of H₂PtCl₆.xH₂O and allylamine.

12. The method of claim 11 further comprising selecting said predetermined molar percentage of said H₂PtCl₆.xH₂O to be between 0.1 and 0.5 mol % and selecting said predetermined molar percentage of allylamine to be between 200 and 400 mol %.

13. A catalytic dehyrdocoupling method for synthesizing metallole copolymers according to the following equation: 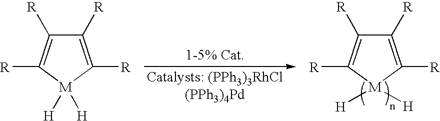
where R is a H or an alkyl or aryl group selected from the group consisting of Me or Ph; and
where M is selected from the group consisting of Si and Ge.

14. A Wurtz coupling polycondensation method for synthesizing metallole copolymers according to the following equations: 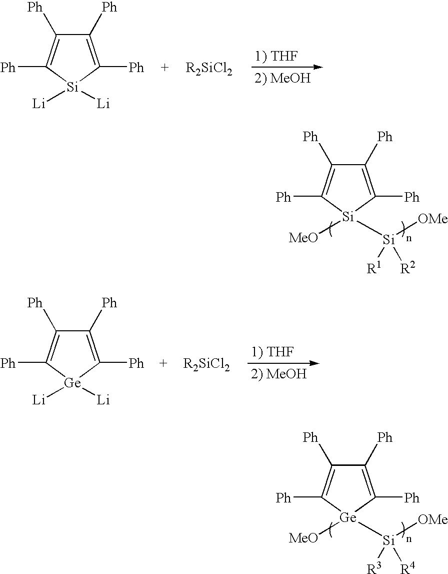
where Ph is a phenyl group, Me is a methyl group, and R is Me or Ph;
where the pair R¹ and R² are selected from the group consisting of: R¹═H and R²═Me; R¹═H and R²═Ph; Rh═Ph and R²═Ph; and R¹═H and R²═H; and
where the pair of R³ and R⁴ are selected from the group consisting of: R³═H and R⁴═Me; R³═H and R⁴=Ph; and R³=Ph and R⁴=Ph.

15. A catalytic dehyrdocoupling method for synthesizing metallole polymers according to the following equation:

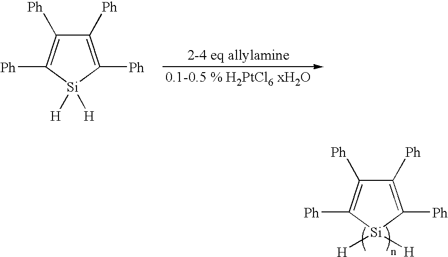

16. A method for detecting an analyte that may be present in ambient air or complex aqueous media comprising:
providing a polymer or copolymer containing a metalloid-metalloid backbone;
exposing said polymer or copolymer to a suspected analyte or a system suspected of including the analyte; and
measuring a quenching of photoluminescence of the metallole polymer or copolymer exposed to said system.

17. The method of claim 16 further comprising selecting said provided polymer or copolymer to be a polymer or copolymer containing tetraphenylsilole.

18. The method of claim 16 further comprising selecting said provided polymer or copolymer to be a polymer or copolymer containing tetraphenylgermole.

19. The method of claim 16 further comprising selecting a metalloid-metalloid backbone of said provided polymer or copolymer group of Si—Si, Ge—Ge, and Si—Ge.

20. The method of claim 16 wherein said step of providing a polymer or copolymer further comprises casting a thin film of said provided metallole polymer or copolymer.

21. The method of claim 20 further comprising depositing said prepared thin film on a glass substrate.

22. The method of claim 16 wherein said step of exposing said polymer or copolymer includes submerging said polymer or copolymer in an aqueous solvent.

23. The method of claim 16 wherein said step of exposing said polymer or copolymer includes submerging said polymer or copolymer in an organic solvent.

24. The method of claim 16 further comprising dissolving the polymer or copolymer in an organic solvent from the group consisting of toluene or THF.

25. The method of claim 16 wherein said step of exposing said polymer or copolymer includes submerging said polymer or copolymer in aqueous inorganic acids.

26. The method of claim 25 further comprising selecting said aqueous inorganic acids from the group consisting of H₂SO₄ and HF.

27. The method of claim 16 wherein said step of measuring a quenching of photoluminescence includes subjecting said polymer or copolymer to fluorescence spectrometry.

28. The method of claim 16 wherein said step of providing a polymer or copolymer comprises dissolving the polymer or copolymer in solution.

29. The method of claim 16 wherein said step of providing a polymer or copolymer comprises producing a colloid of the polymer or copolymer.

30. An inorganic polymer sensor for detecting nitroaromatic compounds comprising:
a substrate; and
a thin film of a metallole polymer or copolymer deposited on said substrate.

31. The sensor of claim 30 wherein said substrate is glass.

32. The sensor of claim 30 wherein said metallole polymer or copolymer is represented by the structure 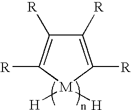
where R is an alkyl group selected from the group consisting of H, Me, or Ph; and
where M is selected from the group consisting of Si and Ge.

33. The sensor of claim 30 wherein said metallole polymer or copolymer is represented by the structure 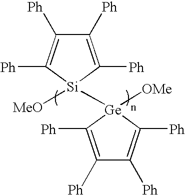
where Ph is a phenyl group and Me is a methyl group.

34. The sensor of claim 30 wherein said metallole polymer or copolymer is represented by the structure 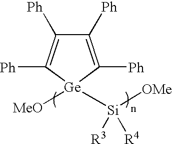
where Ph is a phenyl group and Me is a methyl group; and
where the pair of R³ and R⁴ are selected from the group consisting of: R³═H and R⁴=Me; R³═H and R⁴=Ph; and R³=Ph and R⁴=Ph.

35. The sensor of claim 30 wherein said metallole polymer or copolymer is represented by the structure 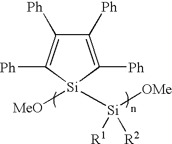
where the pair of R¹ and R² are selected from the group consisting of R¹═H and R²=Me; R¹═H and R²=Ph; and R¹=Ph and R²=Ph.
